(19) United States
(12) Patent Application Publication
(10) Pub. No.: US 2008/0102045 A1
(43) Pub. Date: May 1, 2008

(54) TOPICAL PREPARATIONS CONTAINING ANTIOXIDANT EXTRACTS OF BROCCOLI, GREEN TEA, AND RED TEA

(76) Inventor: Elisabeth K. Shim, Santa Monica, CA (US)

Correspondence Address: BUCHALTER NEMER, 18400 VON KARMAN AVE., SUITE 800, IRVINE, CA, 92612, US

(21) Appl. No.: 11/867,896

(22) Filed: Oct. 5, 2007

Related U.S. Application Data
(60) Provisional application No. 60/850,249, filed on Oct. 5, 2006.

(51) Int. Cl. A61K 33/32; A61K 33/00; A61K 36/31; A61K 36/82; A61K 8/18; A61P 17/00

(52) U.S. Cl. 424/59; 424/642; 424/617; 424/755; 424/729

(57) ABSTRACT

A topical preparation, which includes sunscreens, UV protective lotions and cosmetics is described which includes, at least one physical agent compound, at least one natural or chemically-derived extract, and at least one additional compound. In addition, methods for preparing these topical preparations are disclosed which include: providing at least one physical agent compound, providing at least one natural or chemically-derived extract, providing at least one additional ingredient, and blending the at least one physical agent compound, the at least one natural or chemically-derived extract and the at least one additional ingredient at one or more appropriate temperatures to form the topical preparation.

This application is a U.S. Utility Application that claims priority to U.S. Provisional Application Ser. No. 60/850,249 filed on Oct. 5, 2006, which is incorporated herein in its entirety by reference.

### FIELD OF THE SUBJECT MATTER

The field of the subject matter is topical preparations, including sunscreens and UV protective lotions, that comprise at least one extract of broccoli, green, red teas or combinations thereof, all of which act as antioxidants.

### BACKGROUND

As sunlight become less shielded by the earth's ozone layer and atmosphere, people are more concerned about the effects of UVA and UVB rays on the skin. Sunlight can contribute to both early skin aging and skin cancer. The production of new and more effective topical preparations, such as sunscreens and protective lotions, has increased with demand. There is evidence that sunscreen may prevent the formation of pre-cancerous skin lesions (actinic keratoses), and squamous cell carcinoma. Both ultraviolet A light (UVA) and ultraviolet B light (UVB) can both cause skin injury. UVA, known as the “tanning” rays, induces skin pigmentation and free radical formation. UVB, known as the “burning” rays, induces skin erythema (redness), and sunburn. It is not known which rays contribute more to skin cancer and skin aging, but it is probably due to both types.

There are two main types of sunscreens, physical agents and chemical agents. Physical sunscreens, such as titanium dioxide and zinc oxide, scatter and reflect UV light. Chemical agents, such as avobenzone, cinnamates, and salicylates are also utilized as sunscreens and these agents absorb UV light.

These sunscreens and protective lotions can take the form of stand-alone lotions used by a consumer on the beach or at a sporting event and sunscreen additives to cosmetics, hair products and moisturizers. In addition, sunscreens and protective lotions are being modified to be utilized by those consumers with sensitive skin, those consumers who sweat (athletes), babies and younger children, etc.

Researchers have discovered that dietary botanicals are useful, if ingested, for the prevention of photocarcinogenesis. A wide variety of botanicals, mostly dietary flavonoids or phenolic substances, have been reported to possess substantial anticarcinogenic and antimutagenic activities because of their antioxidant and antiinflammatory properties. The researchers in this study suggest adding these botanicals as dietary supplements for the protection of photocarcinogenesis, whereas these botanicals may favorably supplement sunscreens protection and may provide additional antiphotocarcinogenic protection including the protection against other skin disorders caused by solar UV radiation. (Baliga, Manjeshwar S; Katiyar, Santosh K., Photochem. Photobiol. Sci, Vol. 5 (2), pp. 243-53 (2006)) As stated though, these dietary botanicals are designed for ingestion, and many consumers may not want to consume or may have difficulty in consuming additional supplements each day, (See also: Kramata, P.; Lu, Y. P.; Lou, Y. R.; Cohen, J. L.; Oloha, M,; Liu, S.,; Conney, A. H., Carcinogenesis, Vol. 26 (11), pp. 1965-1974 (2005) Lu, Y. P.; Lou, Y. R.; Liao, J.; Xie, J. G.; Peng, Q. Y.; Yang, C. S.; Connev, A. H., Carcinogenesis, Vol. 26 (8), pp. 1465-1472 (2005))

Ideally, a topical preparation, such as a sunscreen or protective lotion, should have the following qualities: a) shields skin from both UVA and UVB rays, b) is appropriate for all skin types, including sensitive skin, younger skin and older skins c) is derived from natural sources, such as plants and fruits, and d) is able to be blended with a variety of cosmetic preparations in order to produce a full line of skin care products.

### SUMMARY OF THE SUBJECT MATTER

A topical preparation, which includes sunscreens, UV protective lotions and cosmetics is described that includes: at least one physical agent compound, at least one natural or chemically-derived extract, and at least one additional compound.

In addition, methods for preparing these topical preparations are disclosed which include: providing at least one physical agent compound, providing at least one natural or chemically-derived extract, providing at least one additional ingredient, and blending the at least one physical agent compound, the at least one natural extract and the at least one additional ingredient at one or more appropriate temperatures to form the topical preparation.

### DETAILED DESCRIPTION

Recently, there is a developing trend towards adding antioxidants, such as Vitamin E, to skin care preparations to aid in preventing free radical damage from ultraviolet light. There are several studies showing the benefits and the disadvantages of using and/or ingesting Vitamin E, especially with men. Therefore, other antioxidants have been reviewed and surprisingly, novel physical sunscreen agents have been developed and are described herein.

A sunscreen and/or protective lotion has been developed that comprises at least two of the following qualities: a) shields skin from both UVA and UVB rays, b) is appropriate for all skin types, including sensitive skin, younger skin and older skin, c) is derived from natural sources, such as plants and fruits, and d) is able to be blended with a variety of cosmetic preparations in order to produce a full line of skin care products.

A topical preparation, which includes sunscreens, UV protective lotions and cosmetics is described which includes: at least one physical agent compound, at least one natural extract, and at least one additional compound. In some embodiments, contemplated preparations have a SPF or Sun Protection Factor of at least 10. In other embodiments, contemplated preparations have a SPF of at least 15. In yet other embodiments, contemplated preparations have a SPF of at least 25.

Specifically, a physical agent sunscreen is described herein that comprises at least one physical agent compound and at least one natural or chemically-derived extract. These natural or chemically-derived extracts act as antioxidants in the sunscreen. Antioxidants, such as the ones described herein, have been demonstrated in laboratories to have protective effects against UV radiation and show promise in preventing skin cancers and skin aging. In combination with a physical sunscreen, these antioxidants may provide a unique benefit.

In some embodiments, the at least one natural or chemically-derived extract comprises extracts from the cabbage family, such as broccoli extracts and extracts from plants and leaves, such as tea leaves. Contemplated tea leaves which may be utilized are green tea and red tea leaf extracts, and in some cases, black tea.

Sulphorane, a compound found in broccoli extract, has recently been shown in mice to help prevent skin cancer formation after topical application. (Dinkova-Kostova AT, 2005) In one study, 30 mice were treated with UV light (30 mJ/cm2) twice a week for 20 weeks. Subsequently, they received topical broccoli extract on their backs 5 days a week for 11 weeks. This study found that mice who received broccoli extract at a higher dose (1.0 mumol), had a significant reduction in tumor burden, incidence, and multiplicity of about 50% Though the difference between the lower dose of broccoli extract (0.3 mumol) and the controls were not statistically significant, the group receiving the broccoli extract had a lower tumor burden.

Other antioxidants, such as green tea extract (Camelia sinensis) and red tea (Rooibos) extract, have also been found to have beneficial properties regarding free radical scavenging. Green tea extract, orally, has been shown in mice to decrease tumor incidence and tumor burden in a dose-dependent manner (Wang Z Y, 1992). These mice were exposed to cancer-causing chemicals such as 12-O-tetradecanoylphorbol-13-acetate (TPO) and 7-12 dimethylbenz(a)anthracene (DMBA), and ultraviolet light. Green tea extract decreased tumor incidence and formation, and also decreased UVB-induced erythema. Topically, it is has been shown that polyphenol epigallocatechin-3-gallate (EGCG), the effective ingredient in green tea extracts, decreases nitric oxide formation (Song XZ).

Other mechanisms of ant-tumor activity may be that green tea applied topically in mice, induces cytotoxic T cells and inhibits angiogenesis (Mantena SK, 2005). EGOG inhibits matrix metalloproteinases (MMP) 2 and 9, which are thought to contribute to tumor growth and metastasis, while increasing tissue inhibitor of MMP-1 (TIMP-1), which inhibits MMP activity. In addition, EGCG inhibits vascular endothelial growth factor (VEGF), and decreases expression of CD31, a marker for endothelial cells (Mantena SK, 2005).

Furthermore, another study demonstrated that extracts of green tea and rooibos (red tea) also decreased tumor formation in mice (Marnewick J, 2005). Prior to receiving the carcinogens TPO and DMBA, the mice received topically applied extracts of green tea and rooibos. The green tea and rooibos formulations inhibited tumor formation, though green tea performed better.

Therefore, it appears that there is suggestive evidence that extracts of broccoli, green tea, and red tea (rooibos) may be beneficial if added to sunscreen, in order to augment photoprotection against free radical damage, skin aging, and skin cancers.

The at least one physical agent compound comprises those previously mentioned, such as zinc oxide, titanium dioxide or a combination thereof. The physical agent is designed to potentially act—in part or whole—in concert with or synergistically with the at least one natural extract to help protect the skin from sun damage.

At least one additional ingredient may be added to the topical preparation in order to a) facilitate activity of the physical and/or natural extract on the skin or in the product, b) produce a cosmetic product that is appropriate for a specific age group or skin type, c) produce a cosmetically elegant feel to the product, and/or d) produce a specific cosmetic product not typically considered one that might contain a sunscreen, such as eye shadows, blushes, hair products or foundation. These additional ingredients may include water, disodium EDTA, xanthan gum, glycols, glycerin-based compounds, silicates, hydroxides, phosphates, alcohols, benzoates, siloxanes, acids, alumina, citrates, triglycerides, acetates, stearates, polyacrylates, polyisobutenes, polysorbates, glycolates or a combination thereof. Many of these compounds and polymers, along with others that are indeed contemplated, are described in the Examples Section.

A physical sunscreen containing zinc oxide and titanium dioxide, in combination with extracts of broccoli, green tea, and red tea, is to be marketed as “Verdure Matte Moisturizing Sunscreen with Antioxidants SPF 28”. Other included antioxidants include Vitamin E and Willowherb extract.

In addition, methods for preparing these topical preparations are disclosed which include: providing at least one physical agent compound, providing at least one natural extract, providing at least one additional ingredient, and to blending the at least one physical agent compound, the at least one natural extract and the at least one additional ingredient at one or more appropriate temperatures to form the topical preparation.

The at least one physical agent compound, the at least one natural extract and/or the at least one additional ingredient may be provided by any suitable method, including a) buying at least some of the at least one physical agent compound, the at least one natural extract and/or the at least one additional ingredient from a supplier; b) preparing or producing at least some of the at least one physical agent compound, the at least one natural extract and/or the at least one additional ingredient in house using chemicals provided by another source and/or c) preparing or producing at least some of the at least one physical agent compound, the at least one natural extract and/or the at least one additional ingredient in house using chemicals also produced or provided in house or at the location.

As mentioned, the at least one physical agent compound, the at least one natural extract and the at least one additional ingredient are blended at one or more appropriate temperatures to for the topical preparation. As used herein, the term “blended” is an all encompassing term that describes mixing, blending, agitating, integration, incorporation, combining or otherwise merging various components and ingredients contemplated herein.

With respect to the temperature, the Examples Section describes one contemplated embodiment where the first part of the preparation is heated to a temperature of about 78-80° C. and then subsequently cooled as more ingredients are added. It is contemplated that in other embodiments, some ingredients may be blended at or below room temperature, subsequently heated in order to add additional ingredients or induce chemical reactions between some or all components and then finally cooled back to room temperature. It should be understood by one of ordinary skill in the art that heating and cooling steps do not necessarily need to be in any specific order or utilized at all depending on the additional ingredients.

### EXAMPLES

### Example 1

### Moisturizing Lotion (SPF-25)

This Example shows the ingredients for and how to prepare a sunscreen-based moisturizing lotion according to the disclosure presented herein. It should be understood to those of ordinary skill in the art, however, how to make other cosmetic preparations that include the extracts disclosed herein. The Sequence ingredients are found in Table 1 below.

Manufacturing Procedure:

1. In main beaker, combine Sequence #1 ingredients and heat to 78° C.-80° C. with homogenizer mixing.
2. Combine Sequence #2 ingredients and heat to 80° C. with propeller mixing. Mix until uniform.
3. Slowly add Sequence #1 to Sequence #2 with homogenization and mix for 15 minutes.
4 Switch to moderate speed propeller mixing and begin cooling the batch.
5. At 40° C., add Sequence #3 ingredients. Mix well after each addition and continue cooling.
6. At 25° C., add Sequence #4 ingredients, mixing well after each addition.
7. Add Sequence #5 ingredients and mix well, increasing mixing speed as necessary.

8. Mix for an appropriate time until the batch is smooth and uniform.

TABLE 1

| SEQUENCE | PERCENT | INGREDIENT | INCI NAME |
|---|---|---|---|
| 1 | 11.21 | Deionized Water | Water |
| 1 | 0.10 | Dissolvine Na2T | Disodium EDTA |
| 1 | 7.00 | Keftrol (1%) | Xanthan Gum |
| 1 | 1.00 | Botanistat PF-64 | Phenoxyethanol (and) Caprylyl |
| | | | Glycol (and) |
| | | | Ethylhexylglycerin (and) Hexylene |
| | | | Glycol |
| 1 | 4.50 | Liponic EG-1 | Glycereth-26 |
| 1 | 12.00 | Veegum HV (4%) | Magnesium Aluminum Silicate |
| 1 | 0.09 | NaOH (18%) | Sodium Hydroxide |
| 2 | 2.00 | Liponate PE-810 | Pentaerythrityl |
| | | | Tetracaprylate/Tetracaprate |
| 2 | 3.00 | Sensanov WR | C20-22 Alkyl Phosphate (and) C20- |
| | | | 22 Alcohols |
| 2 | 4.00 | Liponate NEB | C12-15 Alkyl Benzoate |
| 2 | 6.00 | Botanisil CP-33 | Cyclopentasiloxane |
| 2 | 1.50 | Jeesilc IDD | Isododecane (and) Dimethicone |
| | | | Crosspolymer-3 |
| 2 | 27.00 | KFS40M170 | Caprylyl Methicone (and) Titanium |
| | | | Dioxide (and) |
| | | | Cyclopentasiloxane (and) C12-15 |
| | | | Alkyl Benzoate |
| | | | (and) Alumina (and) |
| | | | Polyhydroxystearic Acid (and) |
| | | | Methicone |
| 2 | 3.60 | CM3K50X24 | Zinc Oxide (and) |
| | | | Cyclopentasiloxane (and) |
| | | | PEG-10 Dimethicone (and) |
| | | | Methicone |
| 2 | 2.50 | Dracorin GOC | Glyceryl Oleate Citrate (and) |
| | | | Caprytic/Capric |
| | | | Triglyceride |
| 2 | 0.80 | Softisan 378 | Caprylic/Capric/Myristic/Stearic |
| | | | Triglyceride |
| 2 | 0.05 | Vitamin E | Tocopheryl Acetate |
| | | Acetate | |
| 2 | 1.00 | Polyolprepolymer-15 | PEG-8/SMDI Copolymer |
| 2 | 0.50 | Lipopeg 100-S | PEG-100 Stearate |
| 3 | 0.25 | Gorgonian Extract GC | Caprylic/Capric Triglyceride (and) |
| | | | Sea |
| | | | Whip Extract |
| 3 | 0.50 | Emeressence 1160 | Phenoxyethanol |
| 3 | 4.00 | Dragaxat 89 | Ethylhexyl Isononanoate |
| 4 | 1.00 | Fucogel 1000NI | Biosaccharide Gum-1 |
| 4 | 2.00 | SM-2000 | Mica (and) Silica |
| 4 | 0.50 | Willowherb Extract | Water (and) Epilobium |
| | | | Angustifolium Extract |
| 4 | 0.15 | Rooibos Red Tea | Water (and) Butylene Glycol (and) |
| | | | Aspalanthus |
| | | Extract (10%) | Linearis Leaf Extract |
| 4 | 0.50 | Green Tea Extract BG | Butylene Glycol (and) Water (and) |
| | | | Camellia Sinensis Leaf Extract |
| 4 | 0.25 | Broccoli Extract | Water (and) Butylene Glycol (and) |
| | | | Brassica |
| | | | Oleracea italica (Broccoli) Extract |
| 5 | 2.00 | Sepiplus 400 | Polyacrylate-13 (and) Polyisobulene |
| | | | (and) Polysorbate 20 |
| 5 | 1.00 | Hydagen CMF | Chitosan Glycolate |

Specifications:
pH: 7.24 ± 0.2
Viscosity: LVT#4 @ 3 rpm 50,000-60,000 cps
Total cost $8.70/lb

The following list provides typical suppliers of the above-referenced extracts and additives:

| | |
|---|---|
| Dissolvine Na2T | Akzo Nobel |
| Keltrol | Kelco |
| Botanistat PF-64 | D-D Chemco |
| Botanisil CP-33 | |
| Polyolprepolymer-15 | |
| Fucogel 1000NI | |
| Liponic EG-1 | Lipo Chemicals |
| Liponate PE-810 | |
| Liponate NEB | |
| Lipopeg 100-S | |
| Gorgonian Extract GC | |
| Veegum HV | RT Vanderbilt |
| Sensanov WR | Seppic |
| Sepiplus 400 | |
| Jeesilc IDD | Jeen International |
| KFS40M170 | Kobo Products |
| CM3K50X24 | |
| Dracoric GOC | Symrise |
| Dragoxat 89 | |
| Softisan 378 | Sasol |
| Vitamin E Acetate | BASF |
| Emeressence 1160 | Cognis |
| Hydagen | |
| SM-2000 | Presperse |
| Willowherb Extract | Technical Art of Science |
| Green Tea Extract BG | Premier |
| Rooibos Red Tea Extract 10% | |
| Broccoli Extract | |

### Example 2

### Moisturizing Lotion (SPF-28)

This Example shows the ingredients for and how to prepare a sunscreen-based moisturizing lotion according to the disclosure presented herein. It should be understood to those of ordinary skill in the art, however, how to make other cosmetic preparations that include the extracts disclosed herein. The Sequence ingredients are found in Table 2 below. The Manufacturing Procedure is the same as that for Example 1.

TABLE 2
Components of Example 2 Formulation:

| Component | Amount (Weight Percent) |
|---|---|
| Purified Water | 29.7257 |
| Versene NA2/Dissolvine NA2 | 0.1000 |
| Keltrol | 0.1000 |
| Botanistat PF-84 | 1.0000 |
| Liponic EG-1 | 4.5000 |
| Veegum HV | 0.4800 |
| Calistic Soda Beads | 0.0162 |
| Liponate PE-810 | 2.0000 |
| Sensanov WR | 3.0000 |
| Liponate NEB | 4.0000 |
| Botanisil CP-33 | 6.0000 |
| Jeesilc IDD | 1.5000 |
| KFS40M170 (TiO₂ 31.5%) + 5% OVR | 27.000 |
| CM3K50X24 (ZnO₂ 48%) + 5% OVR | 3.8281 |
| Dracoric GOC | 2.5000 |
| Softisan 378 | 0.8000 |
| Vitamin E Acetate | 0.0500 |
| Polyolprepolymer-15 | 1.0000 |
| Lipopeg 100-S | 0.5000 |
| Emeressence 1160 | 0.5000 |
| Dragoxat 89 | 4.0000 |
| Fucogel 1000NI | 1.0000 |
| SM-2000 | 2.0000 |
| Wiilowherb Extract | 0.5000 |
| Rooibos Red Tea Extract (10%) | 0.1500 |
| Actiphyte of Green Tea GL50 | 0.5000 |
| Actiphyte of Broccoii BG50 | 0.2500 |
| Sepiplus 400 | 2.0000 |
| Marine Biopolymer Grade HV | 0.0100 |
| Glycolic Acid 70% | 0.0054 |
| Purified Water | 0.9846 |

### REFERENCES

Dinkova-Kostova A T, Jenkins S N, Fahey J W, Ye L, Wehage S L, Liby K T, Stephenson K K, Wade K L, Talalay P. Protection against UV-light-induced skin carcinogenesis in SHK-1 high risk mice by sulphorane-containing broccoli sprout extracts. Cancer Lett, 2005 Nov. 2, Epub ahead of print.
Wang Z Y, Huang M T, Ferraro T, Wong C Q, Lou Y R, Reuhl K, Iatropoulous M, Yang C S, Conney A H. Inhibitory effect of green tea in the drinking water on tumorigenesis by ultraviolet light and 12-O-tetradecanoylphorbol-13-acetate in the skin of SKH-1 mice. Cancer Res, 1992.52 (5): 1162-70.
Song X Z, Bi Z G, Xu A E. Green tea polyphenol epigallocatechin-3-gallate inhibits the expression of nitric oxide synthase and generation of nitric oxide induced by ultraviolet B in HaCaT cells. Chin Med J (Engl), 2006. 119 (4): 282-7.
Mantena S K, Meeran S M, Elmets C A, Katiyar S K. Orally administered green tea polyphenols prevent ultraviolet radiation-induced skin cancer in mice through activation of cytotoxic T cells and inhibition of angiogenesis in tumors. J Nutr, 2005. 135 (12): 2871-7.
Mantena S K, Roy A M, Katiyar S K. Epigallocatechin-3-gallate inhibits photocarcinogenesis through the inhibition of angiogenic factors and activation of CD8+ T cells in tumors. Photochem Photobiol, 2005. 81(5): 1174-9.
Marnewick J, Joubert E, Joseph S, Swanevelder S, Swar P, Gelderblom W. Inhibition of tumour promotion by extracts of rooibos (Aspalathus linearis) and honeybush (Cyclopea intermedia), unique South African teas. Cancer Lett, 2005, 224 (2): 193-202.

Thus, specific embodiments and applications of topical preparations containing antioxidant extracts of broccoli, green tea and red tea have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

I claim:

1. A topical preparation, comprising:
at least one physical agent compound,
at least one natural or chemically-derived extract, and
at least one additional compound, wherein the at least one physical agent compound, the at least one natural extract, and the as least one additional compound are blended to form the topical preparation.

2. The topical preparation of claim 1, wherein the physical agent comprises zinc oxide, titanium dioxide or a combination thereof.

3. The topical preparation of claim 1, wherein the preparation comprises at least two natural extracts.

4. The topical preparation of claim 1, wherein the preparation comprises at least three natural extracts.

5. The topical preparation of one of claims 1, 3 or 4, wherein the natural or chemically-derived comprises extracts from broccoli, red tea, green tea, black tea or a combination thereof.

6. The topical preparation of claim 1, wherein the preparation is considered a sunscreen or UV protective lotion.

7. The topical preparation of claim 1, wherein the preparation has a SPF of at least 15.

8. The topical preparation of claim 7, wherein the preparation has a SPF of at least 25.

9. The topical reparation of claim 1 comprising:
a combination of zinc oxide and titanium dioxide,
a combination of broccoli extract, red tea extract and green tea extract, and
at least one additional ingredient.

10. The topical preparation of claim 9, wherein the preparation is a sunscreen, UV protective lotion or cosmetic preparation.

11. The topical preparation of claim 1, wherein the preparation comprises a pH of less than about 7.3.

12. The topical preparation of claim 1, wherein the preparation comprises a viscosity of less than about 60000 cps.

13. A method of preparing a topical preparation, comprising:
providing at least one physical agent compound,
providing at least one natural or chemically-derived extract,
providing at least one additional ingredient, and
blending the at least one physical agent compound, the at least one natural or chemically-derived extract and the at least one additional ingredient at one or more appropriate temperatures to form the topical preparation.

14. The method of claim 13, wherein the appropriate temperature comprises blending at a temperature of less than about 80° C.

15. The method of claim 14, wherein the appropriate temperature comprises blending at a temperature of less than about 80° C. followed by gradual cooling.
